SEQUENCE LISTING (1 sequence)

SEQ ID NO: 1 - DNA, 528 bp, Artificial Sequence
  Nucleic acid sequence of the whole GluR-P2A-GluR2 biological entity
atgttaatcgagttctgctacaaatcccgtagtgaatccaagcggatgaagggtttttgtttgatcccacagcaatccatcaacgaagccatacggacatcgaccctcccccgcaacagcggggcaggagccagcagcggcggcagtggagagaatggtcgggtggtcagccatgacttccccaagtccatgcaatcgattccttgcatgagccacagttcagggatgcccttgggagccacgggattggccacaaacttctctctgctaaagcaagcaggtgatgttgaagaaaaccccggccctagcaacgttgctggagtattctacatccttgtcgggggccttggtttggcaatgctggtggctttgattgagttctgttacaagtcaagggccgaggcgaaacgaatgaaggtggcaaagaatgcacagaatattaacccatcttcctcgcagaattcacagaattttgcaacttataaggaaggttacaacgtatatggcatcgaaagtgttaaaatttag

(19) United States
(12) Patent Application Publication
(10) Pub. No.: US 2024/0238356 A1
(43) Pub. Date: Jul. 18, 2024

(54) A Biological Entity for Treating Brain Cancer

(71) Applicant: ALIVID LLC, Rumson, NJ (US)

(72) Inventor: David TUKEY, Rumson, NJ (US)

(73) Assignee: ALIVID LLC, (US)

(21) Appl. No.: 18/558,984

(22) PCT Filed: May 4, 2022
(86) PCT No.: PCT/US2022/027684

§ 371 Date: Nov. 3, 2023

Related U.S. Application Data
(60) Provisional application No. 63/183,945, filed on May 4, 2021.

(51) Int. Cl. A61K 35/763; A61K 45/06; A61K 48/00; A61P 35/00; C12N 15/86

(52) U.S. Cl. CPC A61K 35/763; A61K 45/06; A61K 48/0058; A61P 35/00; C12N 15/86; C12N 2710/16632; C12N 2710/16643

(57) ABSTRACT

Disclosed are a biological entity for treating brain cancer, in particular glioma, and a vector including the biological entity. The biological entity is a construct including at least anti-tumor transgenes comprised of at least one GluA knockdown agent, and preferably at least two GluA knockdown agents, and preferably further includes a fusogenic protein, and immune response promotors of an anti-PD-1 antibody, an anti-CTLA-4 antibody and an IL12. The vector comprises a wild-type HSV-1 virus wherein the biological entity replaces the ICP 34.5 gene of the wild HSV-1 virus. The vector comprising wild type HSV-1 virus modified with the biological entity shows little negative effect on neurons while showing positive effect against human glioblastoma cells, both separately cultured and in co-cultures of neuronal cells and glioblastoma cells.

### CROSS-REFERENCE TO RELATED APPLICATIONS

This application is related, and claims priority to U.S. provisional patent application 63/183,945, filed May 4, 2021 and PCT application PCT/US22/27684, filed May 4, 2022, the contents of both of which are fully incorporated herein.

### BACKGROUND

### 1. Field of the Disclosure

The present disclosure relates to a new biological entity for treating brain cancer, in particular glioma.

### 2. Discussion of the Background Art

### Prior Treatments for Glioma

Glioma is the most common form of malignant brain cancer, and its most virulent form, glioblastoma (GBM), is among the deadliest of cancers. The average survival of a GBM patient is just over a year from diagnosis. The current standard of care for first-line glioblastoma, Temodar, increased Overall Survival from 12.1 to 14.6 months vs radiation alone, as set forth in the FDA Full Prescribing Information for Temodar. (see, FIG. 1).

Over the last 20 years, there have been a series of a small trials investigating oncolytic viruses in glioma. In their original conception, oncolytic viruses (OVs) were viruses that were engineered to infect tumor cells but not normal tissue, primarily through deletion of the ICP34.5 gene of herpes simplex type 1 (HSV-1), which HSV-1 needs to withstand immune responses generated by non-tumor cells (see, Mineta, T. et al. “Attenuated multi-mutated herpes simplex virus-1 for the treatment of malignant gliomas.” Nature Medicine 1, no. 9 (September 1995): 938-943). Given the need to administer OVs at the site of the tumor, the first major pharmaceutical effort with an OV was in skin cancer. Biovex/Amgen's Imlygic, an engineered HSV-1 virus, was approved for skin cancer in 2015. Other viruses have also been engineered for anti-cancer purposes, including adenovirus, vaccinia, polio, coxsackie virus, and others. To date, however, only HSV-1 based viruses have demonstrated clear clinical proof of concept for such treatment.

In glioma, no randomized oncolytic virus trials have been conducted. However, the clinical community has been encouraged by evidence of tumor shrinkage and anti-tumor immune responses, seemingly driven by propagation of the OV in some patients. Qualitatively, there is more OV research being conducted in glioma as a percentage of all research compared to other tumors. There are likely several reasons for this: i) the blood-brain barrier imposes challenges on systemic delivery of traditional pharmacological agents, ii) the invasiveness of evaluation procedures makes intratumoral administration more palatable, and iii) the direness of prognoses yields a risk/benefit calculation that allows for intracerebral administration of experimental viruses.

One early-stage trial was published in The New England Journal of Medicine in April 2021. Investigators at the University of Alabama Birmingham treated patients with an OV construct that was created in 1995. (see, Friedman et al. “Oncolytic HSV-1 G207 Immunotherapy for Pediatric High-Grade Gliomas”. N Engl. J Med 2021; 384:1613-1622) (see, FIG. 2).

One company, DNAtrix, is taking an adenovirus OV into a phase 3 in glioblastoma based on a small number of patients in their early phase trial who responded. (see, Lang et al. “Phase I Study of DNX-2401 (Delta-24-RGD) Oncolytic Adenovirus: Replication and Immunotherapeutic Effects in Recurrent Malignant Glioma.” Journal of Clinical Oncology 36, no. 14, 1419-1427 (May 2018)) (see, FIG. 3). And, in June 2021, Japanese health authorities approved the oncolytic virus, Teserpaturev (G474), marketed by Todo and Daiichi Sankyo, for the treatment of malignant glioma.

A breakthrough for the OV field came in November 2020 when Replimune, a company started by inventors of Imlygic, released proof of concept clinical data for their next-generation OV, RP2. Their first-generation candidate, RP1, had seemingly demonstrated efficacy in skin cancer in combination with immune checkpoint inhibitors, but had not demonstrated single-agent activity. RP2 includes checkpoint inhibitor antibodies against PD-1 and CTLA-4 within its construct. Therefore, RP2, with its seemingly increased potency over RP1, is a “loaded OV”—an OV that used the virus as a delivery agent for anti-cancer agents while still being an anti-cancer agent itself (see, Aroldi, F. et al. “Initial results of a phase 1 trial of RP2, a first in class, enhanced potency, anti-CTLA-4 antibody expressing, oncolytic HSV as single agent and combined with nivolumab in patients with solid tumors”. Poster presented 2020 Society for Immunotherapy of Cancer Annual Meeting) (see, FIG. 4). A Replimune (REPL) RP-1 schematic is shown in FIG. 5, (see, Thomas, S. et al., “Development of a new fusion-enhanced oncolytic immunotherapy platform based on herpes simplex virus type 1.” Journal for Immuno Therapy of Cancer 7, no. 1, 6-17 (December 2019)).

The public company Oncorus is currently testing an OV with 5 immune-stimulating transgenes, named ONCR-177 (see, FIG. 6). ONCR-177 payloads were said to be designed to stimulate a de novo productive anti-tumor response, (see, Kennedy, E. M. et al., “Design of ONCR-177 base vector, a next generation oncolytic herpes simplex virus type-1, optimized for robust oncolysis, transgene expression and tumor-selective replication.” Poster presented at American Association for Cancer Research annual meeting 2019)).

### Calcium-Permeable AMPA Receptors (CPARs)

In 2019, Venkataramani, et al. published in Nature that Calcium-permeable AMPA receptors (CPARs) contribute to tumor cell maintenance and spread through their signaling at synapses of neurons onto glioma tumor cells (Venkataramani, V. et al. “Glutamatergic synaptic input to glioma cells drives brain tumour progression.”, Nature 573, 532-538 (2019)) (FIG. 7). CPARs are a subtype of the AMPA-type glutamate receptor that have emerged in the last 10-15 years as significant drivers of change processes in the brain. CPARs most often have been implicated in learning and memory processes, where they are part of the physical manifestation of information storage in the hippocampus and are required for reward-driven behaviors.

The present inventor helped elucidate the intracellular mechanisms by which CPARs are trafficked from the neuronal cytoplasm to the synapse (see, Tukey, D. S. et al., “Sucrose ingestion induces rapid AMPA receptor trafficking.” Journal of Neuroscience 33, No. 14, 6123-6132 (April 2013)) (FIG. 8), and demonstrated that CPARs themselves can be drivers of change once they are incorporated into synapses, (see, Tukey, D. S. and Ziff, E. B., “Ca2+-permeable AMPA (α-amino-3-hydroxy-5-methyl-4-isoxazolepropionic acid) receptors and dopamine D1 receptors regulate GluA1 trafficking in striatal neurons”, Journal of Biological Chemistry 288, No. 49, 35297-35306 (December 2013)) (FIG. 9). Importantly, CPARs are also integral to changes at synapses between neurons and glial cells (see, Ge, W P et al., “Long-term potentiation of neuron-glia synapses mediated by A2+-permeable AMPA receptors.” Science 312, No. 5779, 1533-1537 (June 2006)). Glial cells are neuronal support cells whose genetic dysregulation gives rise to glioma.

### SUMMARY

The present disclosure is a new biological entity for treating brain cancer, namely glioma. The present disclosure applies neuronal synapse biology using a new oncolytic virus (OV).

The present disclosure provides a new OV that is a herpes simplex virus 1 (HSV-1) engineered to infect and replicate selectively in tumor cells while expressing transgenes that stimulate anti-tumor immune responses to prevent, or at a minimum mitigate, the spread of brain cancer by blocking neuron-tumor synapses. The new OV is an HSV-1 having its ICP 34.5 gene deleted and replaced with a construct that may include anti-tumor transgenes and is driven by the ICP47 gene promoter.

The new OV is administered by intratumoral injection to achieve a better overall response rate, and thus likely a better overall survival rate, than the current standard of care/treatment. The current standard of care is temozolomide alone, and other OVs currently in development for glioma, discussed above. An improvement in overall response rate slowing the spread of brain cancer, is an immeasurable benefit to a patient's life.

The new OV preferably contains at least genetic interference of the proteins GluA1 and GluA2, primary subunits of AMPA receptors including CPARs, in glioma cells to slow proliferation of tumor cells by interfering with the trafficking of AMPA receptors and therefore the glioma cell-neuron synapse. The new OV is designed to deliver GluA1 and GluA2 knockdown agents in an efficient way of achieving local synaptic GluA1 and GluA2 knockdown to augment the efficacy of a loaded oncolytic construct in glioma.

### BRIEF DESCRIPTION OF THE DRAWINGS

Further details, features, and advantages of the disclosure ensue from the following description of exemplary embodiments shown in the Figures.

FIG. 1 shows the Kaplan-Meier curves for overall survival rate of radiation therapy alone versus radiation therapy plus Temodar.

FIG. 2 shows the Kaplan-Meier curves for overall survival rate of patients treated with the OV construct G207, created in 1995.

FIG. 3 shows brain scans of patients A, B, C, with accompanying graphs showing % change in tumor size over time, who responded to an adenovirus oncolytic agent developed by DNAtrix.

FIG. 4 shows antitumor activity and kinetics of response for RP2 oncolytic HSV as single agent and combined with nivolumab in patients with solid tumors.

FIG. 5 shows the schematic for the construct of RP-1.

FIG. 6 shows the design of ONCR-177 base vector, an HSV-1 containing 5 immune-stimulating transgenes within its construct.

FIG. 7a shows representative time series of GB cells (green) and brain micro vessels (red) under control conditions (arrows, 5 independent experiments in 4 mice) versus high dose isoflurane (4 independent experiments in 4 mice); FIG. 7b shows the invasion speed of GB cells (n=254 control cells versus n=143 isoflurane cells in 4 S24 PDX mice); FIGS. 7c and 7d show representative time series of non-responsive and responsive S24 PDX cells, respectively, (neuronal ChR2 stimulation (red lines); 9 independent experiments in 6 mice). Cells measured at 0 hrs. (red arrows) and 5 hrs. (green arrowheads); FIG. 7e shows invasion speed of S 24 PDX GB cells (non-responsive (NR), n=164 cells in 5 mice; responsive (R), n=53 mice;

FIG. 7f shows representative time series showing glioma invasion of cells expressing Glu-2A-DN-GFP with tdTomato (arrows) compared with glioma cells expressing only tdTomato; FIG. 7g shows that a dominant negative GluR2 subunit significantly slows tumor invasion speed in an animal model of malignant glioma; FIG. 7h shows representative images of S 24 xenografts with GB cells expressing Glu-2A-DN-GFP with tdTomato (arrows) or only tdTomato at 0 and 14 days; FIG. 7i shows cell density changes over 14 days (n=13 regions in 5 mice); FIG. 7j shows glioma regions on days 0 and 14 under control conditions and after treatment with the AMPAR antagonist perampanel; FIG. 7k shows cell density on day 14 versus day 0 under control versus perampanel (PER) in two different cell lines S 24 and BG5. FIGS. 7 f and g are prior art believed relevant for the purposes of this disclosure.

FIG. 8 shows electron microscopy showing induction of multistep GluA1 trafficking by sucrose ingestion, thereby demonstrating that prevention of intracellular GluA1 trafficking prevents the strengthening of synapses. In FIG. 8a GluA1 was PEG labelled and particles were classified into 5 post-synaptic regions: (1) intraspinous; (2) extrasynaptic membrane or (3) PSD (cleft, at PSD, near PSD); FIG. 8b shows electron micrographs that were prepared from water, sucrose/water, or sucrose animals (3 animals per test group); FIGS. 8c-f show that repeated sucrose ingestion elevates intraspinous and PSD GluA1 while acute sucrose ingestion induces rapid GluA1 trafficking to the extrasynaptic membrane. Note: in FIGS. 8c-e data are presented as averages of the number of particles per spine.

FIG. 9 shows confirmation of AMPA-induced GluA1 trafficking through genetic blockage of trafficking, suggesting that synapses with CPARs have mechanisms for inducing feed-forward synaptic strengthening.

FIG. 10 shows construction and validation of human GluR1/2 expressing virus. Human gluR1-P2A-gluR2 expressing oncolytic HSV-1 was constructed by driving the expression of the transgenes from ICP47 HSV-1 promoter. To construct HSV-1 expressing hGlur1/2, infectious virus DNA of parental gamma 35.5 double deleted virus was used. Lysates of U20S cells co-transfected with infectious viral DNA and plasmid DNA (with UL26/27 flanking sequence and gluR1/2 and eGFP expressing transgene sequence) was harvested 4 days later for infection of a fresh monolayer and eGFP expression was used to visualize recombinant plaques. PCR (A) from purified viral genomic DNA of 10× plaque purified viral plaque shows the correct band of 528 bp. Immunoblotting (B) shows expressed gluR2 protein in infected SF-295 glioblastoma cells.

FIG. 11 shows a schematic of a vector comprising wild type HSV-1 virus modified with a preferred biological entity according to the present invention, where the biological entity replaces the ICP 34.5 gene of the wild type HSV-1 virus.

FIG. 12 shows the components and description, both preferred embodiments and options, for each element of the biological entity shown in FIG. 11.

FIG. 13 shows the genetic map of the present invention comprising wild type HSV-1 virus modified with the biological entity used in Experiment 1, below.

FIG. 14 shows visually the cytopathic effects of human gluR1/2 expressing HSV-1, used in Experiment 1, below. SF-295 glioblastoma cells infected with 1 Multiplicity of Infection (MOI) of the wild type or gluR1/2 expressing HSV-1 of the present invention. HSV-1 expressing hgluR1/2 causes extensive cytopathic effect in SF-295 cells.

FIG. 15 shows in bar graph form the visual results shown in FIG. 14, specifically a measure of cell viability (metabolism), evaluated using the CellTiter-glo kit (Promega) and fold changes are calculated relative to untreated control cells. Values are mean standard deviation of three independent experiments. ***p<0.0005, ****p<0.00005.

### DESCRIPTION OF THE PREFERRED EMBODIMENTS

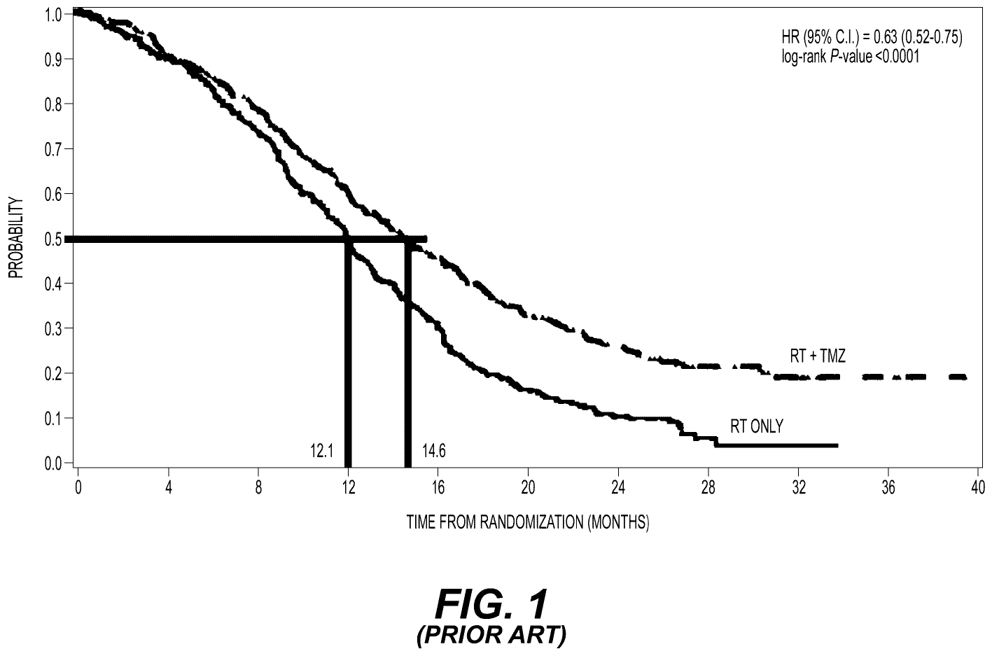

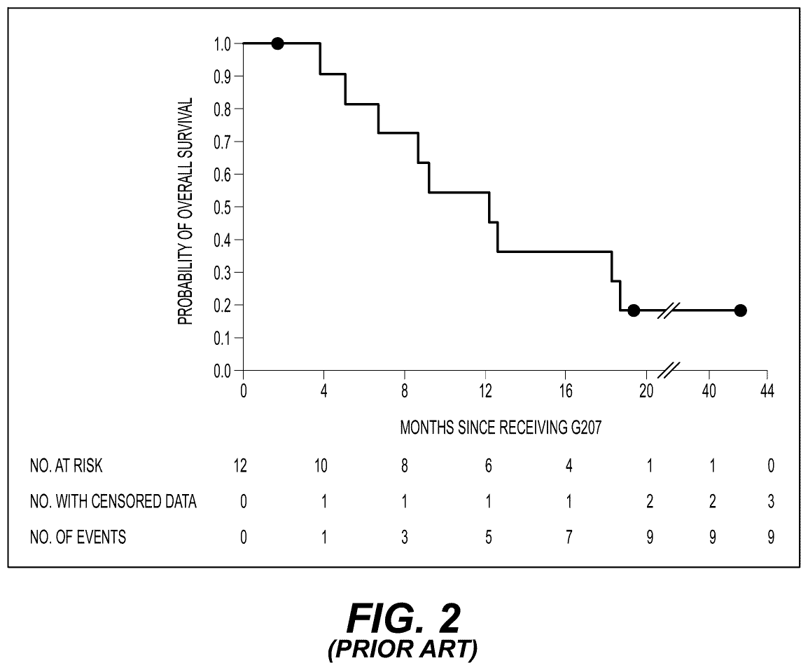

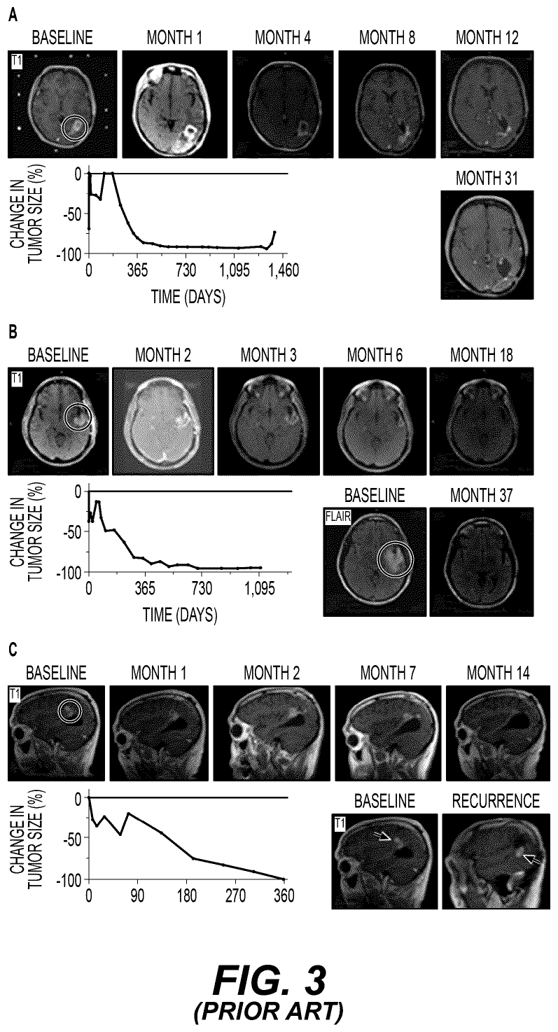

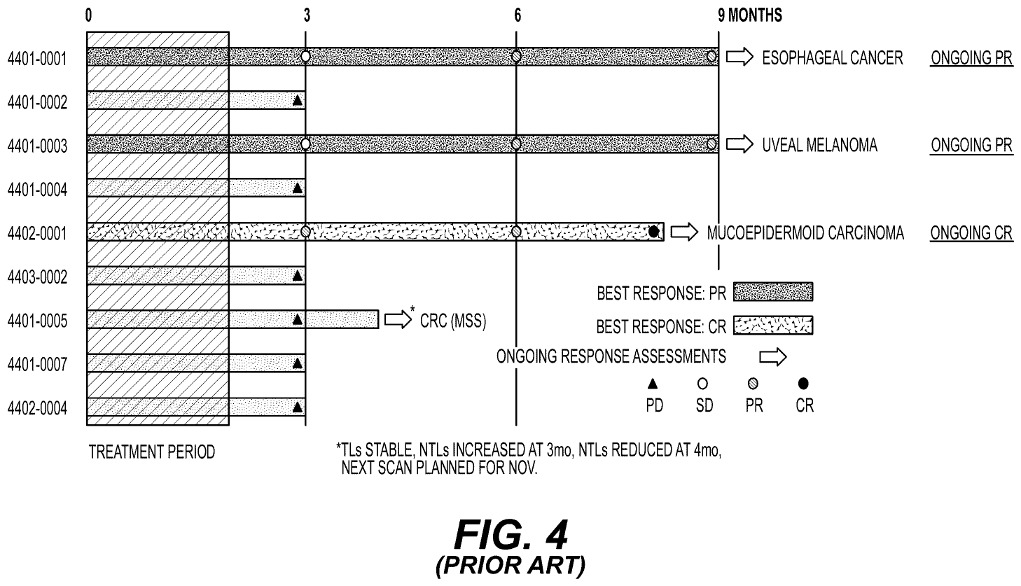

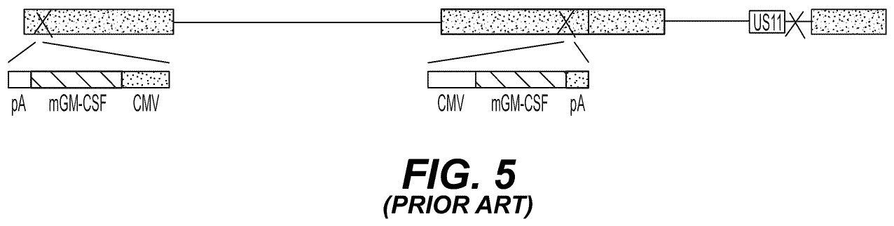

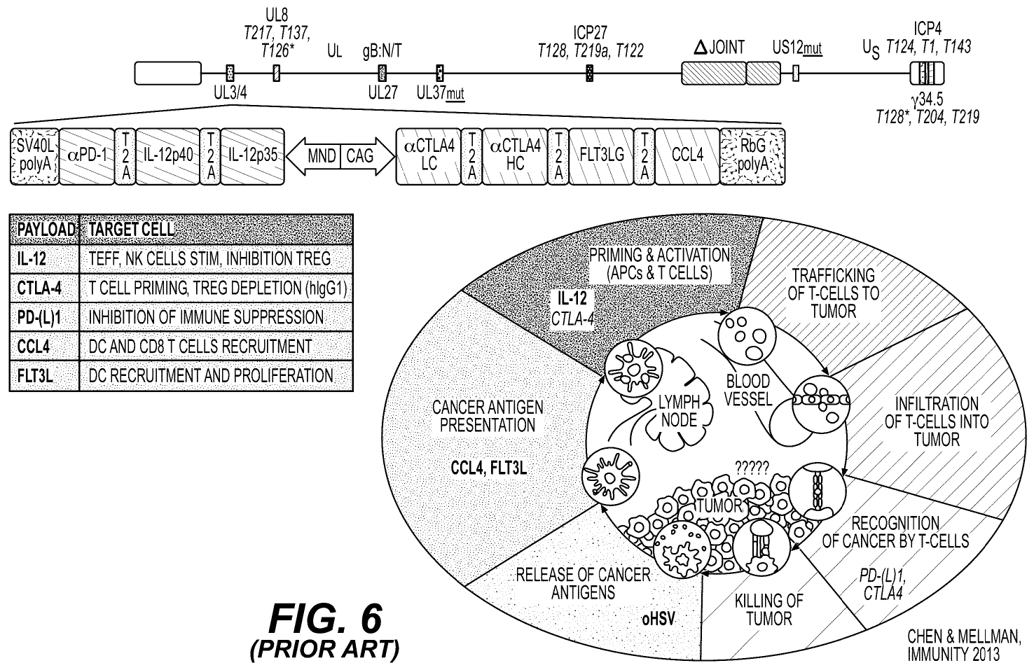

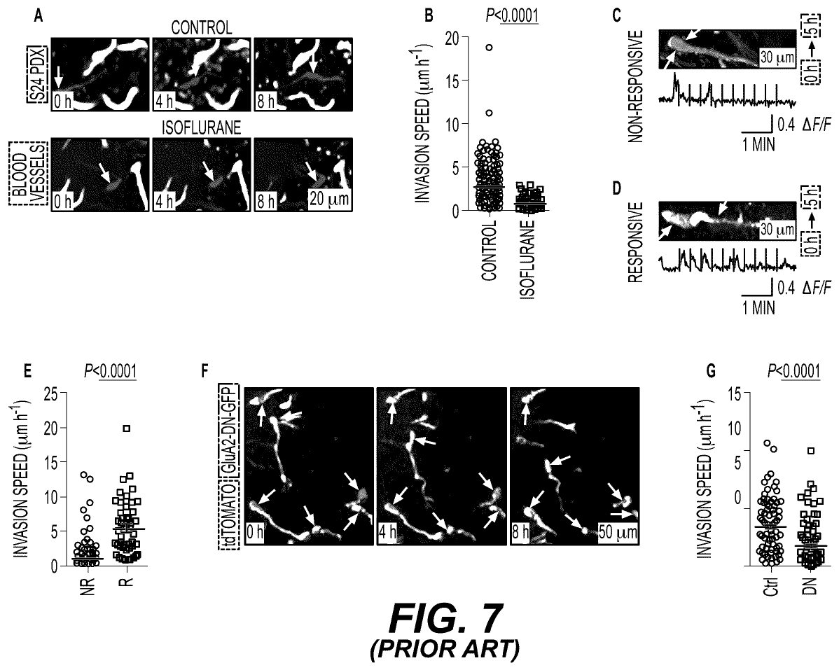

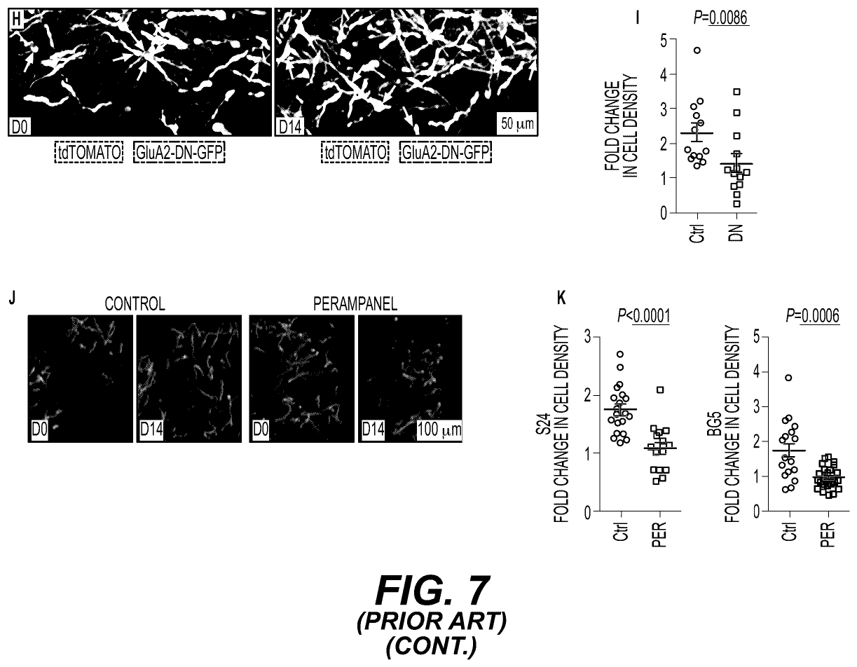

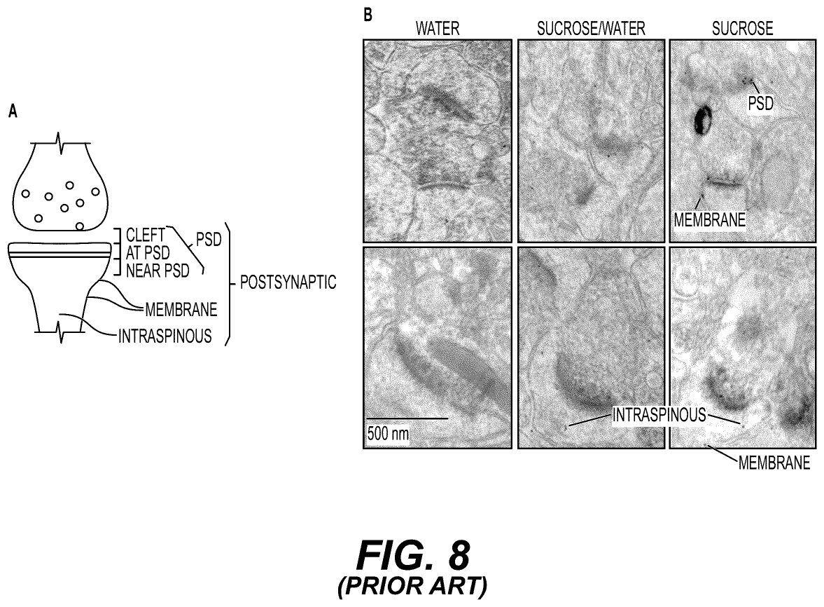

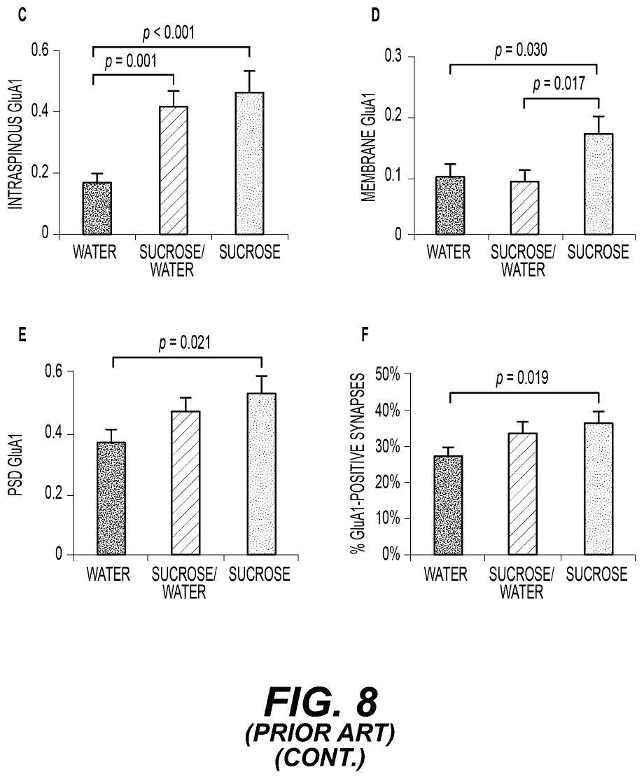

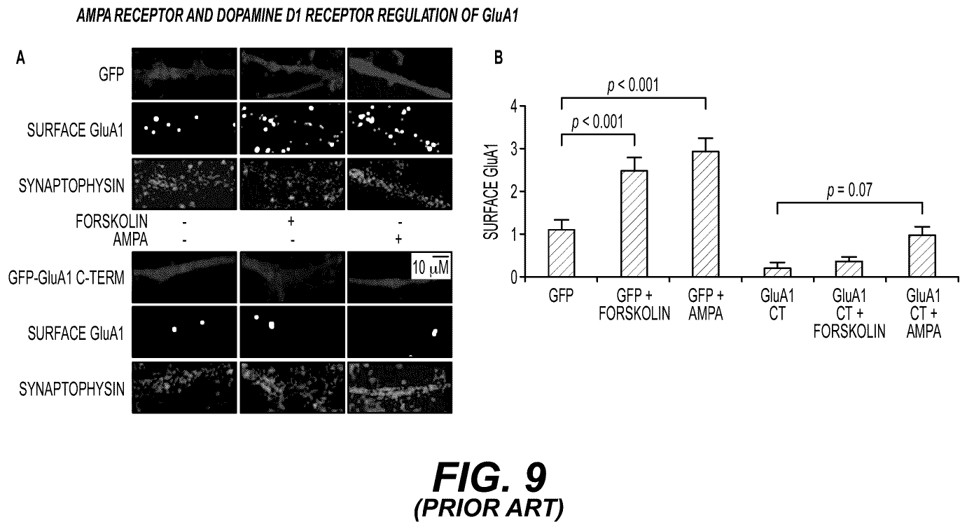

As described above, FIGS. 1-9 illustrate prior art attempts to treat glioma using various models and biologic entities, descriptions of AMPA receptor trafficking pertinent to the present invention. Because those skilled in the art are likely aware of the detail and analysis of these prior art attempts, no detailed description of any of them will be presented here. Those skilled in the art who may not be aware of the details of these prior art attempts can easily review the work described in Figures by surveying the literature identified in the Background of the Invention, above.

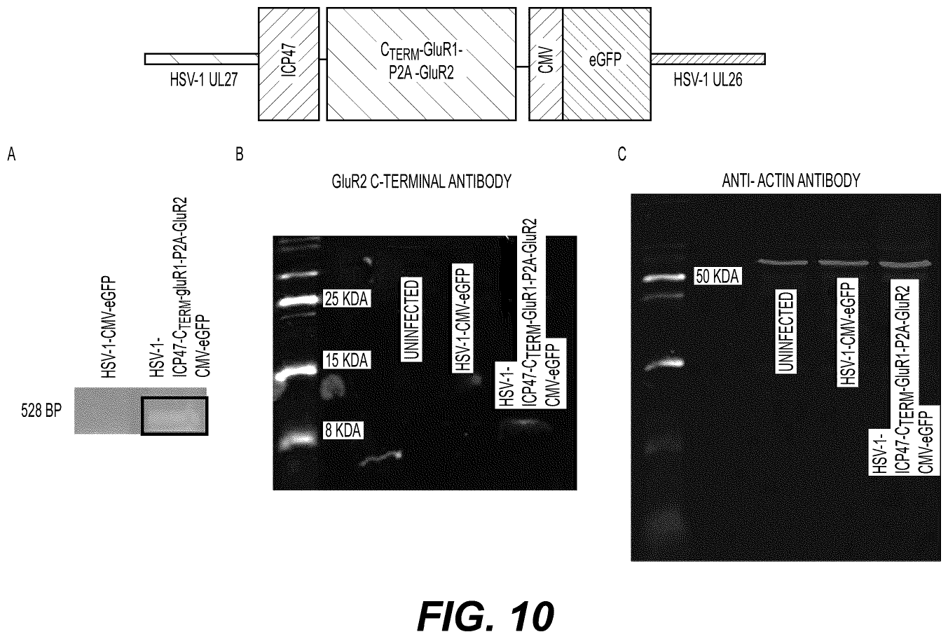

FIG. 10 shows a composition of the present invention, where the HSV-1 ICP34.5 gene is deleted and replaced with a biological entity comprised of the C-termini of the GluR1 and GluR2 AMPA receptor subunits. FIG. 10a shows that the biological entity is intact in the virus. FIGS. 10b and 10c show that the C-terminal of the AMPA receptor subunit GluR2 is expressed specifically by the virus of the present invention.

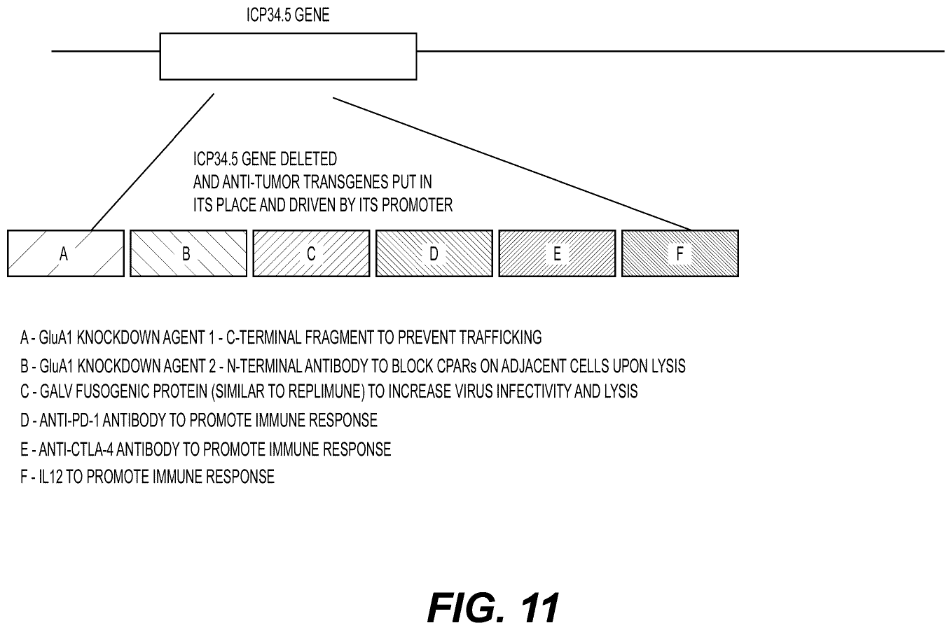

FIG. 11 shows a schematic of a vector comprising wild type HSV-1 virus modified with a biological entity according to the present invention, where the biological entity replaces the ICP 34.5 gene of the wild HSV-1 virus. The ICP 34.5 gene of wild type HSV-1 virus is deleted and replaced with a construct according to FIG. 11 that comprises anti-tumor transgenes that are driven by the ICP 34.5 promotor of the wild HSV-1 virus. The anti-tumor transgenes include two AMPA receptor subunit interference, or GluA knockdown agents. The anti-tumor transgenes preferably further include a fusogenic protein, an anti-PD-1 antibody, an anti-CTLA-4 antibody and an IL 12 construct. The first AMPA receptor subunit interference agent comprises a C-terminal fragment of GluA1 and is designed to prevent trafficking of GluA1 to the neuronal synapse and/or the extrasynaptic membrane. The second GluA knockdown agent could comprises a C-terminal fragment of GluA2 designed to prevent trafficking of GluA2 to the neuronal synapse and/or the extrasynaptic membrane, or it could comprise an N-terminal antibody to GluA1 that could block CPAR synaptic transmission on adjacent cells after tumor cell lysis.

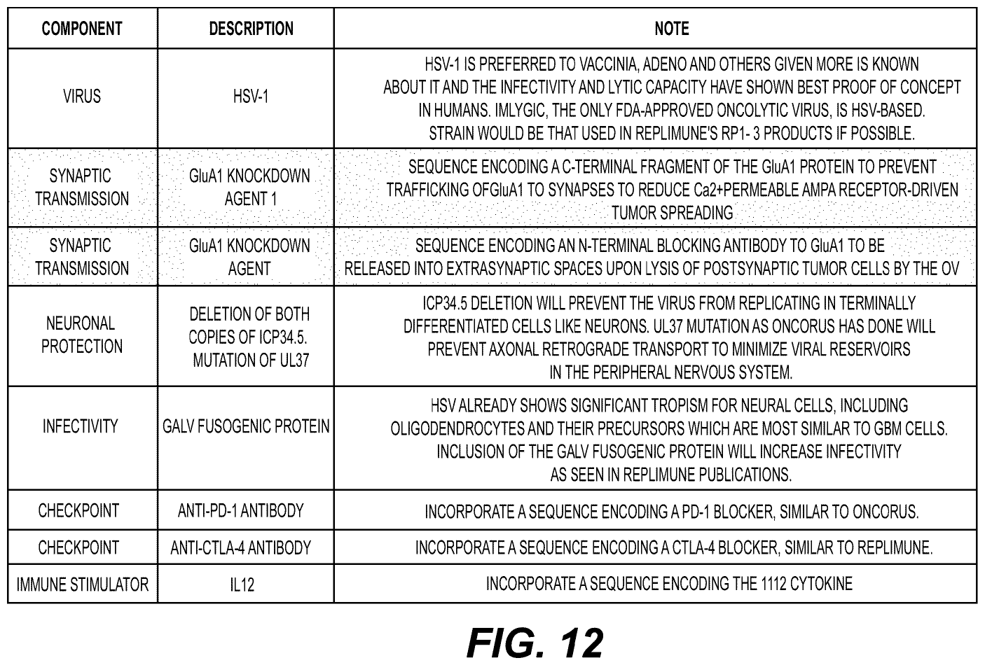

FIG. 12 shows each component and its general and detailed description of a preferred biologic entity of the present invention. FIG. 12 also shows variations and options for each component, including preferred options. In general, an HSV-1 virus is preferred because more is known about it and a strain of it is being used in Replimune's RP1-3 products. The strain used in the RP1-3 products would be the preferred virus to use. As mentioned above, the new OV contains at least genetic knockdown of the proteins GluA1 and GluA2. The first GluA knockdown agent comprises a C-terminal fragment of GluA1 and is designed to prevent trafficking of GluA1 to the neuronal synapse and/or the extrasynaptic membrane. The second GluA knockdown agent comprises a C-terminal fragment of GluA2 and is designed to prevent trafficking of GluA2 to the neuronal synapse and/or the extrasynaptic membrane. For neuronal protection, the deletion of both copies of the ICP34.5 gene is designed to prevent the virus from replicating in terminally differentiated cells like neurons. For infectivity, HSV-1 already shows significant tropism for neural cells, including oligodendrocytes and their precursors which are most similar to GBM cells. Inclusion of the GALV fusogenic protein will increase infectivity. There are preferably provided two checkpoint inhibitors, an anti-PD-1 antibody, preferably a sequence encoding a PD-1 blocker, similar to Oncorus and an anti-CTLA-4 antibody, preferably a sequence encoding a CTLA-4 blocker, similar to Replimune. Finally, there is preferably incorporated an immune stimulator IL12, incorporating a sequence encoding the IL12 cytokine.

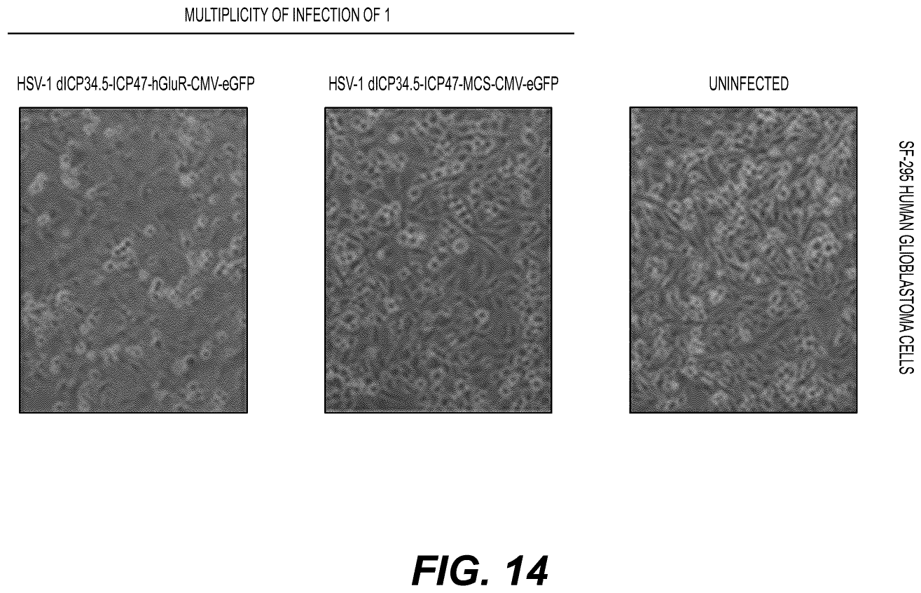

FIG. 14 shows visually the effects of treating plated SF-295 human glioblastoma cells with either vehicle control, wild-type HSV-1, or a vector of HSV-1 containing the biological entity of the present invention.

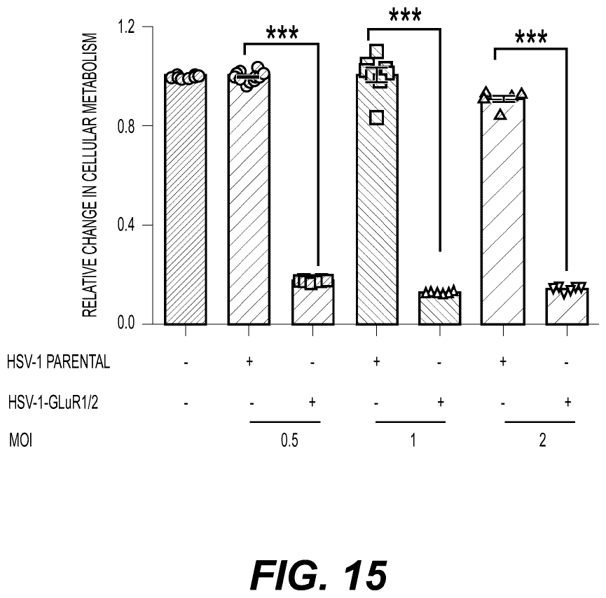

FIG. 15 shows the results of FIG. 14 in bar graph form. In FIG. 15, the “+” and “−” indicate the presence or absence of the indicated virus.

### EXAMPLES

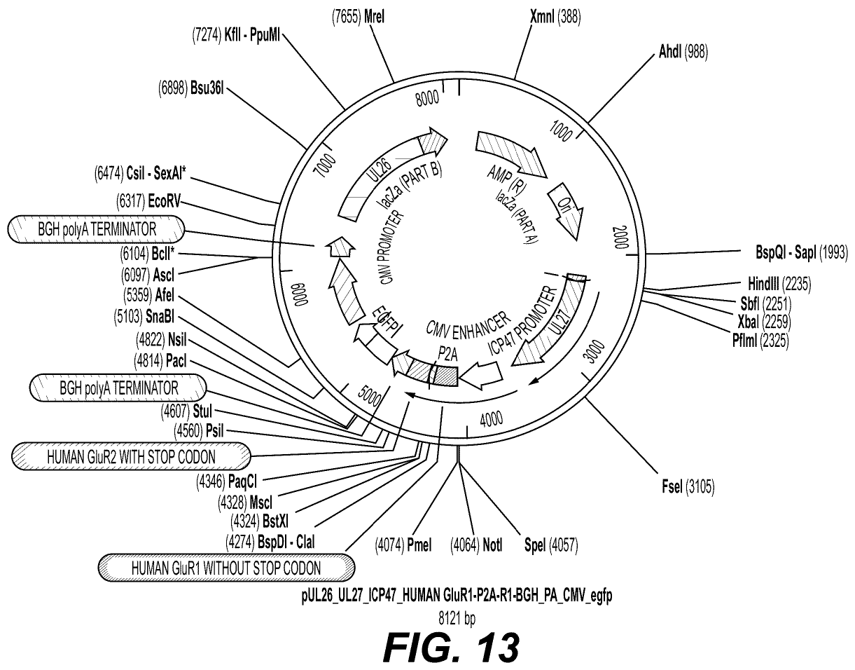

Construction and validation of human GluR1/2 expressing virus for use in the Experiments was performed as follows. Human gluR1-P2A-gluR2 expressing oncolytic HSV-1 was constructed by driving the expression of the transgenes from the ICP47 HSV-1 promoter. (see, FIG. 13). To construct HSV-1 expressing the Glur1/2, infectious virus DNA of parental gamma 34.5 double deleted virus was used. Lysates of U20S cells co-transfected with infectious viral DNA and plasmid DNA (with UL26/27 flanking sequence and gluR1/2 and eGFP expressing transgene sequence) was harvested 4 days later for infection of a fresh monolayer and eGFP expression was used to visualize recombinant plaques. PCR from purified viral genomic DNA of 10× plaque purified viral plaque showed the correct band of 528 bp. Immunoblotting showed expressed gluR2 protein in infected SF-295 glioblastoma cells. The nucleic acid sequence of the whole GluR-P2A-GluR2 biological entity is:

ATGttaatcgagttctgctacaaatcccgtagtgaatccaagcggatga
agggtttttgtttgatcccacagcaatccatcaacgaagccatacggac
atcgaccctcccccgcaacagcggggcaggagccagcagcggcggcagt
ggagagaatggtcgggtggtcagccatgacttccccaagtccatgcaat
cgattccttgcatgagccacagttcagggatgcccttgggagccacggg
attggccacaaacttctctctgctaaagcaagcaggtgatgttgaagaa
aaccccggccctagcaacgttgctggagtattctacatccttgtcgggg
gccttggtttggcaatgctggtggctttgattgagttctgttacaagtc
aagggccgaggcgaaacgaatgaaggtggcaaagaatgcacagaatatt
aacccatcttcctcgcagaattcacagaattttgcaacttataaggaag
gttacaacgtatatggcatcgaaagtgttaaaatttag

### Experiment 1

SF-295 human glioblastoma cells were plated using high glucose DMEM with 10% FBS as the support medium. The duration of this experiment was 2 days. At the end of the experiment, the SF-295 cells proliferated greatly in both the uninfected plated cells and the cells infected with the wild-type HSV-1 virus, showing visually virtually no difference in cellular metabolism between the two (see, FIG. 13). On the other hand, the plated cells infected with the vector of HSV-1 containing the biological entity of the present invention show greatly reduced cellular metabolism compared to the other two plated cells. These visual results were confirmed by calculating the effect of the wild-type HSV-1 containing the ICP 34.5 gene, or the HSV-1 containing GluR1/2 of the present invention replacing the ICP 34.5 gene of the wild-type HSV-1 and presenting these results in bar graph form (see, FIG. 15).

### Experiment 2

Human neurons are plated on two plates using an appropriate medium. To one plate is added the wild-type HSV-1 virus to infect them, while to the other plate is added the vector of HSV-1 containing the biological entity of the present invention. Observations are made at 0, 3, 10 and 21 days. At the end of the 21 days, there is no visual difference between the viability of the human neurons plated on either plate. This shows that the biological entity of the present invention has no visually detectable negative effect on human neurons.

### Experiment 3

Rat postnatal day 1 neurons are grown in appropriate medium for 7 days. At day 7, SF-295 human glioblastoma cells are plated on top of the neurons in ratios of 1:1, 1:2, 1:5 and 1:10 (glioblastoma cells: rat neurons) using an appropriate medium. At 7 days post the addition of the SF-295 cells, the co-cultures are treated with either vehicle control, wild type HSV-1 virus, or HSV-1 virus containing the biological entity of the present invention. Two days post infection, there is a significant decrease in SF-295 invasion speed and cellular metabolism in the co-cultures treated with the virus containing the biological entity of the present invention compared to the co-cultures treated with wild-type virus, and a significant decrease in dendritic spine number and size, as measured by confocal microscopy, in the co-cultures treated with the virus of the present invention compared to wild-type virus.

What is claimed is:

1. A biological entity that is effective in killing human glioblastoma cells comprising at least one anti-tumor transgene comprised of at least one AMPA receptor interference, or GluA knockdown agent.

2. The biological entity according to claim 1, wherein at least one anti-tumor transgene comprises two GluA knockdown agents.

3. The biological entity according to claim 2, wherein the two GluA knockdown agents target AMPA receptor subunits, including GluR1 and GluR2.

4. The biological entity according to claim 1, wherein the biological entity further comprises a fusogenic protein, an immune response promotor in the form of an anti-PD-1 antibody, an anti-CTLA-4 antibody and an IL12 construct.

5. A vector that is effective in killing human glioblastoma cells comprising a wild-type HSV-1 virus and at least one anti-tumor transgene comprised of at least one GluA knockdown agent.

6. The vector according to claim 5, wherein the at least one anti-tumor transgene comprises two GluA knockdown agents.

7. The vector according to claim 6, wherein the two GluA knockdown agents target AMPA receptor subunits, including GluR1 and GluR2.

8. The vector according to claim 5, wherein the anti-tumor transgenes further comprise a fusogenic protein, an immune response promotor of an anti-PD-1 antibody, an anti-CTLA-4 antibody and an IL2.

9. The vector according to claim 4, wherein the vector replaces the ICP 34.5 gene of the wild HSV-1 virus.
